(19) United States
(12) Patent Application Publication
(10) Pub. No.: US 2025/0171772 A1
(43) Pub. Date: May 29, 2025

(54) Genome Wide Screen of RNAi Molecules

(71) Applicant: ELEVEN THERAPEUTICS LTD., Tel Aviv (IL)

(72) Inventors: Yaniv Erlich, Raanana (IL); Gregory James Hannon, Cambridgeshire (GB)

(21) Appl. No.: 18/261,478

(22) PCT Filed: Jan. 16, 2022
(86) PCT No.: PCT/IL2022/050062

§ 371 Date: Jul. 13, 2023

Related U.S. Application Data
(60) Provisional application No. 63/140,881, filed on Jan. 24, 2021.

(51) Int. Cl. C12N 15/10; C12N 15/113

(52) U.S. Cl. CPC C12N 15/1086; C12N 15/1131; C12N 2310/531; C12N 2320/11

(57) ABSTRACT

This disclosure relates to methods, kits and cell lines for retrieving potent RNAi triggers sequences, by utilizing a cell population having an altered RNAi biogenesis machinery, expressing in the cell population nucleic acid constructs, encoding a candidate RNAi trigger targeting a target gene, selecting cells expressing the target gene, rescuing the genetic alteration in the RNAi machinery, and identifying cells expressing a potent RNAi trigger by identifying cells which no longer express the target gene or which express only low levels of the target gene, as compared to the expression level of the target gene prior to rescuing procedure.

### CROSS-REFERENCE TO RELATED APPLICATIONS

This application is the U.S. National Stage of International Patent Application No. PCT/IL2022/050062, filed on Jan. 16, 2022, which claims the benefit of and priority to U.S. Provisional Patent Application No. 63/140,881, filed on Jan. 24, 2021, the contents of each of which are hereby incorporated by reference in their entireties.

### TECHNOLOGICAL FIELD

This disclosure relates to methods, kits and cell lines for retrieving potent RNAi triggers sequences, in particular to finding potent siRNAs, using a dedicated assay.

### BACKGROUND

RNAi has been suggested as an efficient therapeutical modality for various conditions, including, but more limited to, prophylactic treatment of respiratory viruses. For example, an siRNA cocktail against the Spike and NSP12 transcripts of SARS-CoV have been used in prophylactic and therapeutic regimens against SARS coronavirus in Rhesus macaque.

However, of the main challenges of siRNA-based prophylactic therapy is the cost of goods. Unlike most vaccines and small molecule-based drugs, siRNA synthesis is considerably more expensive, and the global supply chain of GMP-grade siRNA drugs limited.

There therefore is a need for finding hyper-potent siRNA molecules that can substantially reduce the required dosage and, by that, the cost of goods. Such high-potent molecules would also have a subsidiary outcome of a smaller likelihood for off-target effects and a better safety profile, which is a must for any, let alone prophylactic, medical treatment.

### SUMMARY

There is provided herein a system and a method (also referred to herein as “SensAI” and “SensAI assay”) enabling large scale evaluation of RNAi triggers against desired RNA targets, such as, but not limited to, against SARS-CoV-2 transcripts. These RNAi triggers can be then converted to siRNA molecules.

Advantageously, and as opposed to other strategy of finding hyper-potent siRNA molecules, SensAI enables a thorough and cost-effective evaluation of various siRNA triggers against targets, which enables identification of potent molecules, after only one round of sequencing, saving major time and effort (less than the typically required 2 months or more).

As a further advantage, the SensAI assay provides a high-throughput evaluation of the same RNAi trigger against a substantial number of targets to quantify the bandwidth of the RNAi trigger. For example, in some cases, such as in the case of viral transcripts, it is highly desired that the RNAi trigger will not only repress a specific target against which it was designed, but also that it be able to act against additional homologous targets derived from close/similar viral strains. In other instances, such as in the case of “gain of function” genetic diseases (e.g. Huntington), it is highly desired to have RNAi triggers with very narrow bandwidth, ensuring that the siRNA will target the mRNA transcript that carries the pathological mutation, but not the wild-type transcript. By simultaneously testing a large number of RNAi triggers-target pairs (including targets with imperfect complementarity), SensAI allows to find the siRNA that are both potent and have the bandwidth suiting the application.

According to some embodiments, there is provided a method for retrieving potent RNAi triggers sequences, the method comprising:
a. providing a cell population comprising at least one genetic alteration in the RNAi biogenesis machinery that substantially decreases the maturation of RNAi triggers expressed from the cell's genome;
b. expressing in the cell population a plurality of nucleic acid constructs, each construct comprising:
   a nucleic acid sequence encoding a candidate RNAi trigger potentially targeting a target gene, the down-regulation of which is desired, wherein the plurality of constructs differ in the sequence of the guide strand or target region
c. selecting cells expressing the target gene,
d. rescuing the genetic alteration in the RNAi machinery;
e. identifying cells expressing a potent RNAi trigger by identifying cells which are no longer expressing the target gene or which express only low levels of the target gene, as compared to the expression level of the target gene prior to rescuing procedure.

According to some embodiments, the nucleic acid construct further comprises a nucleic acid encoding the target sequence, wherein the target sequence is transcriptionally fused to a nucleotide sequence encoding a reporter gene. According to some embodiments, the selecting of cells expressing the construct comprises selecting cells expressing the reporter gene; and wherein the identifying of cells which are no longer expressing the target gene comprises identifying cells no-longer expressing the reporter gene or which express only low levels of the reporter gene, as compared to the expression level of the reporter gene prior to rescuing procedure.

According to some embodiments, the method further includes a step of identifying the sequences of the guide strands expressed by the identified cells.

According to some embodiments, the RNAi trigger is an shRNA. According to some embodiments, the guide strands of the plurality of shRNAs overlappingly cover a mRNA or a UTR of a gene of interest.

According to some embodiments, the method further includes a step of selecting suitable guide strands by discarding sequences that cannot be synthesized well, sequences that have sequence attributes that are typically associated with poor shRNA response, sequences whose guide shRNA seed region can potentially match a human transcript or any combination thereof.

According to some embodiments, the construct further comprises a unique barcode sequence.

According to some embodiments, the target exhibits partial complementarity to the guide sequence.

According to some embodiments, the expressing of the construct comprises cloning the construct into a lentiviral vector and infecting the cells with the lentiviral vector. According to some embodiments, the target multiplicity of infection is below 15%.

According to some embodiments, the genetic alteration in the RNAi biogenesis is a Dicer deletion and wherein rescuing the genetic alteration in the RNAi machinery comprises expressing a dicer gene in the cell.

According to some embodiments, the exogenous Dicer is a modulated Dicer. According to some embodiments, the modulated Dicer comprises a destabilizing domain (ddDicer). According to some embodiments, the method further includes a step of treating the cells expressing the ddDicer with a predetermined concentration of Shield-1 molecules, thereby at least partially stabilizing the ddDicer.

According to some embodiments, the method further includes treating the cells with a predetermined concentration of an shRNA targeting the exogenous Dicer, so as to controllably reduce Dicer activity in the cells.

According to some embodiments, after selecting cells with a potentially strong RNA trigger, the method further comprises:
introducing a same or a different genetic alteration into the identified cells to thereby substantially decrease the expression of the RNAi triggers,
selecting cells that express the target gene,
rescuing the same or the different the genetic alteration, and
identifying cells expressing a potent RNAi trigger by identifying cells which no longer express the target gene or which express only low levels of the target gene, as compared to the expression level of the target gene prior to the rescuing procedure.

According to some embodiments, there is provided a kit comprising:
a. a first vector comprising
   i. a nucleic acid sequence encoding a candidate RNAi trigger potentially targeting a target sequence, the down-regulation of which is desired; and
   ii. a nucleic acid sequence encoding the target sequence, wherein the target sequence is transcriptionally fused to a nucleotide sequence encoding a reporter gene;
   iii. a second vector comprising a nucleic acid sequence encoding an exogenous Dicer.

According to some embodiments, the exogenous Dicer is a modulated Dicer having impaired functionality.

According to some embodiments, the kit further includes a molecule configured to stabilize the modulated Dicer, thereby restoring Dicer functionality. According to some embodiments, the molecule is Shield-1.

According to some embodiments, the kit further includes a Dicer siRNA.

According to some embodiments, the target sequence comprises at least part of an mRNA or a 3′UTR of a target gene.

According to some embodiments, the target sequence comprises at least part of a 3′UTR of a target gene.

According to some embodiments, the target sequence targets a SARS-COV2 transcript.

According to some embodiments, the first vector further comprises two PCR annealing sites flanking the shRNA and the target gene respectively.

According to some embodiments, the shRNA is a mir-30 based shRNA.

According to some embodiments, the reporter gene is a florescent reporter gene.

According to some embodiments, the guide strand is devoid of Adenine at position 20.

According to some embodiments, the first vector is a lentiviral vector.

According to some embodiments, the lentiviral vector comprises a selection marker configured to allow selection of cells infected with the lentiviral vector.

According to some embodiments, there is provided a cell line expressing:
a. a nucleic acid sequence encoding a candidate RNAi trigger comprising a guide strand potentially targeting a target sequence, the down-regulation of which is desired; and
b. a nucleic acid sequence encoding the target sequence, wherein the target sequence is transcriptionally fused to a nucleotide sequence encoding a reporter gene;
   wherein the cell line comprises a genetic alteration in the RNAi biogenesis machinery.

According to some embodiments, the genetic alteration in the RNAi biogenesis is a Dicer deletion.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

The invention will now be described in relation to certain examples and embodiments with reference to the following illustrative figures so that it may be more fully understood.

FIG. 1 shows the negative strand sgRNA1 including all structural proteins and the leader peptide. Its 5′ position starts from the right most region. Various proteins in the SARS-CoV-2 genome are indicated.

FIG. 2A illustratively depicts a SensAI construct including two PCR annealing sites, a mir-30 based shRNA with a guide and passenger strand, a spacer with cloning sites (or a reporter gene), and a 50 bp region that recapitulates a target site within a genomic context.

FIG. 2B illustratively depicts a lentiviral vector including a SensAI construct.

FIG. 3 schematically illustrates the screening procedure of the hereindisclosed SensAI assay.

FIG. 4A shows logistic curves of 10 candidate siRNAs, obtained by interpolating the median mCherry/eGFP ratio under different shRNA concentrations via a logistic curve (standardized such that the median at 0 pM is 1.0). The IC50 estimate of each siRNA is the concentration at which the median ratio is 50%.

FIG. 4B shows estimated IC50 for the shRNA candidates (log scale). The IC50 estimate of each shRNA is reported above its bar. Error bars denote standard errors.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

For convenience, certain terms used in the specification, examples, and appended claims are collected here. Unless otherwise defined, all technical and scientific terms used herein have the same meaning as commonly understood by one of ordinary skill in the art to which this invention pertains.

As used herein, a “nucleotide” comprises a nitrogenous base, a sugar molecule, and a phosphate group. A nucleic acid may include naturally occurring nucleosides (e.g., adenosine, thymidine, guanosine, cytidine, uridine, deoxyadenosine, deoxythymidine, deoxyguanosine, and deoxycytidine), nucleoside analogues (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, locked nucleic acids, arabinose, and hexose). According to some embodiments, the sugar and/or phosphate groups may be modified to include a peptide bond, so as to obtain a Peptide Nucleotide Acid (PNA).

As used herein, the term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g. by DNA replication or transcription of DNA or RNA, respectively). DNA and RNA can also be chemically synthesized. RNA can be post-transcriptionally modified. The terms “target mRNA” and “target transcript” are synonymous as used herein.

As used herein, the term “RNA interference” (“RNAi”) refers to selective intracellular degradation of RNA (also referred to as gene silencing). As used herein an RNAi molecule may collectively refer to small interfering RNAs and short hairpin RNA.

As used herein, the term “RNAi trigger”, refers to siRNA, microRNA, or shRNA or any other RNA molecule that triggers that RNAi machinery.

As used herein, the term “small interfering RNA” (“siRNA”), also referred to in the art as “short interfering RNAs,” refers to an RNA (or RNA analogue) comprising between about 10-60 or 15-25 nucleotides (or nucleotide analogues) that is capable of directing or mediating RNA interference. Generally, as used herein, the term “siRNA” refers to double stranded siRNA (as compared to single stranded or antisense RNA). In certain embodiments, the 3′ end of the RNAi molecules may include additional nucleotides that create an overhang, such as “TT”.

As used herein, the term “short hairpin RNA” (“shRNA”) refers to an siRNA (or siRNA analogue) precursor that is folded into a hairpin structure and contains a single stranded portion of at least one nucleotide (a “loop”), e.g., an RNA molecule that contains at least two complementary portions hybridized or capable of hybridizing to form a double-stranded (duplex) structure sufficiently long to mediate RNAi (as described for siRNA duplexes), and at least one single-stranded portion, typically between approximately 1 and 10 nucleotides in length that forms a loop connecting the regions of the shRNA that form the duplex portion. The duplex portion may, but typically does not, contain one or more mismatches and/or one or more bulges consisting of one or more unpaired nucleotides in either or both strands. Without wishing to be bound by theory, shRNAs are thought to be processed into siRNAs by the conserved cellular RNAi machinery. shRNAs are capable of inhibiting expression of a target transcript that is complementary to a portion of the shRNA (referred to as the antisense or guide strand of the shRNA). In general, the features of the duplex formed between the guide strand of the shRNA and a target transcript are similar to those of the duplex formed between the guide strand of an siRNA and a target transcript. In certain embodiments of the invention the 5′ end of an shRNA has a phosphate group while in other embodiments it does not. In certain embodiments of the invention the 3′ end of an shRNA has a hydroxyl group.

As used herein, the term “RNAi-inducing vector” includes a vector whose presence within a cell results in transcription of one or more RNAs that self-hybridize or hybridize to each other to form an RNAi molecule. In various embodiments of the invention this term encompasses plasmids, e.g., DNA vectors (whose sequence may comprise sequence elements derived from a virus), or viruses, (other than naturally occurring viruses or plasmids that have not been modified by the hand of man), whose presence within a cell results in production of one or more RNAs that self-hybridize or hybridize to each other to form an RNAi molecule. In general, the vector comprises a nucleic acid operably linked to expression signal(s) so that one or more RNA molecules that hybridize or self-hybridize to form an RNAi molecule is transcribed when the vector is present within a cell. Use of the term “induce” is not intended to indicate that the RNAi agent necessarily activates or upregulates RNAi in general but simply indicates that presence of the vector within a cell results in production of an RNAi agent within the cell, leading to an RNAi-mediated reduction in expression of an RNA to which the agent is targeted.

An RNAi-inducing entity is considered to be targeted to a target transcript for the purposes described herein if (1) the agent comprises a strand that is substantially complementary to the target transcript over 15-29 nucleotides, e.g., 15, more preferably at least about 17, yet more preferably at least about 18 or 19 to about 21-23 or 24-29 nucleotides. For example, in various embodiments of the invention the agent comprises a strand that has at least about 70%, preferably at least about 80%, 84%, 89%, 90%, 91%, 92%, 93%, 94%, 95%, 96%, 97%, 98%, 99%, or 100% precise sequence complementarity with the target transcript over a window of evaluation between 15-29 nucleotides in length, e.g., over a window of evaluation of at least 15, more preferably at least about 17, yet more preferably at least about 18 or 19 to about 21-23 or 24-29 nucleotides in length; or (2) one strand of the RNAi agent hybridizes to the target transcript under stringent conditions for hybridization of small (<50 nucleotide) RNA molecules in vitro and/or under conditions typically found within the cytoplasm or nucleus of mammalian cells.

As used herein the term “target” “target gene” and “target site” may be used interchangeably and may be any transcript, the downregulation of which is desired. According to some embodiments, the target site may be a human transcript. According to some embodiments, the target site may be a transcript of a pathogen, such as, but not limited to, a virus.

As used herein, the term “guide strand” refers to the strand of the siRNA which is incorporated into the RNA-induced silencing complex (RISC) and which causes degradation of the transcript to which it pairs.

As used herein, the term “the passenger strand” is the strand of the siRNA complementary to the guide strand and which is degraded when the two strands separate.

As used herein, the term “complementary” refers to the capacity for precise pairing between particular bases, nucleosides, nucleotides or nucleic acids. For example, adenine (A) and uridine (U) are complementary; adenine (A) and thymidine (T) are complementary; and guanine (G) and cytosine (C), are complementary and are referred to in the art as Watson-Crick base pairings. If a nucleotide at a certain position of a first nucleic acid sequence is complementary to a nucleotide located opposite in a second nucleic acid sequence, the nucleotides form a complementary base pair, and the nucleic acids are complementary at that position. One of ordinary skill in the art will appreciate that the nucleic acids are aligned in antiparallel orientation (i.e., one nucleic acid is in 5′ to 3′ orientation while the other is in 3′ to 5′ orientation). A degree of complementarity of two nucleic acids or portions thereof may be evaluated by determining the total number of nucleotides in both strands that form complementary base pairs as a percentage of the total number of nucleotides over a window of evaluation when the two nucleic acids or portions thereof are aligned in antiparallel orientation for maximum complementarity. According to some embodiments, if the window of evaluation is 15-16 nucleotides long, substantially complementary nucleic acids may have 0-3 mismatches within the window; if the window is 17 nucleotides long, substantially complementary nucleic acids may have 0-4 mismatches within the window; if the window is 18 nucleotides long, substantially complementary nucleic acids may contain 0-5 mismatches within the window; if the window is 19 nucleotides long, substantially complementary nucleic acids may contain 0-6 mismatches within the window. In certain embodiments the mismatches are not at continuous positions. In certain embodiments the window contains no stretch of mismatches longer than two nucleotides in length. In preferred embodiments a window of evaluation of 15-19 nucleotides contains 0-1 mismatch (preferably 0), and a window of evaluation of 20-29 nucleotides contains 0-2 mismatches (preferably 0-1, more preferably 0). In addition, according to some embodiments, the positions of the mismatches within the RNAi triggers play an important role. For example, in miRNA mismatches outside the second to seventh positions from the 5′ end of the RNAi trigger (known as “the seed region”) may be tolerated in RNAi triggers that mimic or harness microRNA like repression.

According to some embodiments, the RNAi molecules disclosed herein may be purified. Purification of the nucleic acids described herein may include, but is not limited to, nucleic acid clean-up, quality assurance and quality control. Clean-up may be performed by methods known in the arts such as, but not limited to, AGENCOURT.RTM. beads (Beckman Coulter Genomics, Danvers, Mass.), or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

As used herein, the terms “Dicer” “endoribonuclease Dicer” and “helicase with RNase motif” may be interchangeably used and refer to an enzyme, part of the RNase III family, that in humans is encoded by the DICER1 gene. Dicer cleaves double-stranded RNA and pre-microRNA into short double-stranded RNA fragments termed small interfering RNA and microRNA, respectively.

As used herein, the term “position effect variegation” and PEV refer to variation in expression of a genetic element caused by it being located in an unexpressed region, e.g. the silencing of a gene through its juxtaposition with heterochromatin.

As used herein, the terms “single-stranded positive-strand virus” or “single-stranded positive-strand RNA virus”, refer to a virus having a genome of either “positive” (also referred to as “plus”) strand RNA, i.e. protein is translated either directly from the viral genome or from RNA intermediaries having the same polarity as the corresponding viral mRNA.

As used herein, the term “SARS-CoV-2” is directed to a pleomorphic RNA virus of the Corona genus Coronavirus in the Coronaviridae. When infecting humans, the SARS-CoV-2 virus may result in the COVID-19 condition.

As used herein, the term “prevent infection” and “prophylactic” may refer to siRNAs capable of reducing the chance of a non-infected individual being infected with a virus, e.g. SARS-CoV-2 when encountering an infected subject. For example, preventing infection of a caregiver attending to an infected subject.

As used herein, the term “reducing/attenuating/preventing spread” refers to RNAi triggers capable of reducing the chance of an infected subject infecting another subject during an encounter. As a non-limiting example, preventing spread may include preventing an infected patient from infecting others. As another non-limiting example, preventing spread may include preventing a caregiver being infected, optionally without knowledge thereof (e.g. due to the infection being asymptomatic or during latency) from spreading the infection to others.

As used herein, the term “reducing viral load” refers to an optionally temporary reduction in the number of viral particles at least in treated tissues, such as nasal or buccal tissues.

As used herein, the terms “approximately” or “about” in reference to a number are generally taken to include numbers that fall within a range of 5% or in the range of 1% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

As used herein, the singular forms “a,” “an” and “the” include plural referents unless the context clearly dictates otherwise.

As used herein, “optional” or “optionally” means that the subsequently described event or circumstance does or does not occur, and that the description includes instances where said event or circumstance occurs and instances where it does not.

According to some embodiments, there is provided a method/system (also referred to herein as “SensAI” and “SensAI assay”) enabling large scale evaluation of RNAi triggers against a desired target gene/transcript.

According to some embodiments, the method comprises choosing for synthesis a plurality of oligos (also referred to herein as “SensAI constructs”), each oligo comprises a “targeting sequence” targeting a transcript the downregulation of which is desired.

According to some embodiments, each of the plurality of SensAI constructs include a targeting sequence targeting a different, overlapping regions of the one or more RNA targets. As a non-limiting example, each SensAI constructs may include a sequence configured to pair with overlapping sequences (targeted sequences) within the SARS-COV2 genome.

According to some embodiments, the choosing of suitable shRNAs comprises a step of discarding/filtering out targeting sequences that cannot be synthesized well, sequences that have sequence attributes that are typically associated with poor shRNA response, sequences whose guide shRNA seed region can potentially match a human transcript or any combination thereof. Each possibility is a separate embodiment.

According to some embodiments, the step of discarding/filtering further comprises discarding/filtering out sequences that are not conserved among related species, the attenuation, treatment, preventing infection of which is likewise desired (e.g. close viral strains).

According to some embodiments, each of the plurality of SensAI constructs have a length of 120-220, or 150-200 nucleotides (nt). As a non-limiting example each of the plurality of SensAI constructs have a length of 185 nt.

According to some embodiments, each of the plurality of SensAI constructs include two PCR annealing sites, preferably of 10-25 nt or 15-20 nt (e.g. 18 nt).

According to some embodiments, each of the plurality of SensAI constructs include a mir-30 based shRNA with a guide and passenger strand including the targeting sequence.

According to some embodiments, each of the plurality of SensAI constructs also include a 30-100 bp, e.g. about 50 bp that recapitulates/encompasses the target site (the targeted sequence), i.e. the sequence aimed to be downregulated by the shRNA, if efficient (also referred to herein as the “targeted sequence”).

The targeted sequence is part of a transcript (mRNA or UTR) of a gene of interest.

Typically, the targeting sequence comprises about 22 nucleotides, as siRNA or shRNA molecules that are processed by the RNAi machinery eventually result in a 22 nucleotide guide sequence in the RISC complex.

According to some embodiments, each of the plurality of SensAI constructs include a spacer region with cloning sites. According to some embodiments, the cloning sites may be EcoRI or Mlul restriction sites. According to some embodiments, the spacer region may include the reporter gene. According to some embodiments, the reporter gene may be cloned into the spacer region.

According to some embodiments, the targeted sequence is located within the 3′UTR of the reporter gene so as not to compromise the function of the reporter; however, other positions within the reporter are also possible provided that the reporter remains functional.

Non-limiting examples of suitable reporters include: acetohydroxyacid synthase (AHAS), alkaline phosphatase (AP), beta galactosidase (LacZ), beta glucoronidase (GUS), chloramphenicol acetyltransferase (CAT), various fluorescent proteins (e.g., green fluorescent protein (GFP) and its variants; red fluorescent protein and its variants, yellow fluorescent protein and its variants, such as VENUS, etc.), luminescent proteins (e.g., horseradish peroxidase (HRP) and luciferase), nopaline synthase (NOS), octopine synthase (OCS), and derivatives thereof. Reporters may also be those that confer resistance to a drug, such as neomycin, ampicillin, bleomycin, chloramphenicol, gentamycin, hygromycin, kanamycin, lincomycin, methotrexate, phosphinothricin, puromycin, doxycycline, and tetracyclin. Reporters can also be lethal genes, such as herpes simplex virus-thymidine kinase (HSV-TK) sequences, as well as sequences encoding various toxins including the diphtheria toxin, the tetanus toxin, the cholera toxin and the pertussis toxin. A further negative selection marker is the hypoxanthine-guanine phosphoribosyl transferase (HPRT) gene for negative selection in 6-thioguanine. In addition, reporters can encode cell surface antigens. Any protein expressed on the cell surface is suitable, with CD4 and CD8 being particularly preferred.

According to some embodiments, the reporter gene is a florescent reporter, such as, but not limited to, Venus, GFP, RFP, mCherry etc.

According to some embodiments, the SensAI constructs further includes a second reporter serving as an indicator of successful infection when the reporter construct is packaged into a virion by a producer or packaging cell via an appropriate packaging signal (i.e., “ψ” signal) located on the reporter construct. Often, the packaging signal is located immediately downstream of the 5′LTR when the reporter construct is based on a retroviral vector backbone. Preferably, when the second reporter serves as an indicator of infection, the second reporter is a selection gene such that cell selection can be based on survival that is dependent upon the selection gene.

According to some embodiments, each of the plurality of oligos include a unique barcode sequence of 5-10 bp (e.g. 6 bp).

According to some embodiments, the method further comprises cloning each of the plurality of oligos into a vector, such as, but not limited to, a lentiviral vector and subsequently infecting cells (at a target multiplicity of infection ensuring low chance of more than one lentiviral integration per cell—typically below 15%) and evaluating their reporter gene expression. If an shRNA is potent, it will result in low reporter gene expression and thus a dim fluorescent signal of the cell. Poorly potent shRNA, on the other hand, will not be able to target the transcript and the cells will have a bright fluorescent signal.

It is understood to one of ordinary skill in the art that a possible complication is “position effect variegation”, resulting, for example, in constructs with poorly potent shRNAs being due to them being integrated into a region with diminished expression and thus in low reporter gene expression, being identified as potent (false positive).

Advantageously, the herein disclosed method/system overcomes this deficiency by infecting cells (e.g. 293T cells) that are Dicer^{null/null} with the SensAI constructs. The absence of Dicer prevents the maturation of the shRNA, meaning that reporter gene expression is initially independent of shRNA potency. This enables an initial selection of cells with high reporter gene expression, i.e. cells in which the locus of integration is active (undergoes expression).

Once cells expressing the construct are selected, the expression of Dicer in these cells is restored, e.g. using a synthetic construct.

According to some embodiments, the Dicer expression is a modulated Dicer expression quantitively coupling the potency of the shRNA to the strength of the reporter signal. Without being bound by any theory, the modulated Dicer expression provides an RNAi machinery in which hyper-potent shRNAs will still be able to inhibit their targets, while other shRNA will cause little or no inhibition.

According to some embodiments, the modulated Dicer expression is obtained by expressing a modulated Dicer such as, but not limited to, a Dicer which includes a destabilizing domain (ddDicer), based on a mutant FKBP12 protein. The destabilizing domain targets the fused protein to the proteasome, but it is possible to rescue the fused protein using Shield-1, a highly permeable, non-toxic small molecule that binds to FKBP12 and stabilizes it. Using this strategy, the expression of Dicer may be tittered by introducing different levels of Shield-1.

Alternatively, increasing concentrations of siRNA against Dicer may be utilized to regulate Dicer expression.

The following examples are included to demonstrate examples of certain preferred embodiments of the invention. It should be appreciated by those of skill in the art that the techniques disclosed in the examples that follow represent approaches the inventors have found function well in the practice of the invention, and thus can be considered to constitute examples of preferred modes for its practice. However, those of skill in the art should, in light of the present disclosure, appreciate that many changes can be made in the specific embodiments that are disclosed and still obtain a like or similar result without departing from the spirit and scope of the invention.

### EXAMPLES

The following examples exemplifies the use of the herein disclosed SensAI in finding efficient siRNAs against SARS-COV2. It is however understood by one of ordinary skill in the art that SARS-COV2 targeting serves as an example only, and that the SensAI assay may be used for determining the siRNA potency of shRNAs designed against any target.

### Example 1—Designing a SensAI Library for SARS-CoV-2

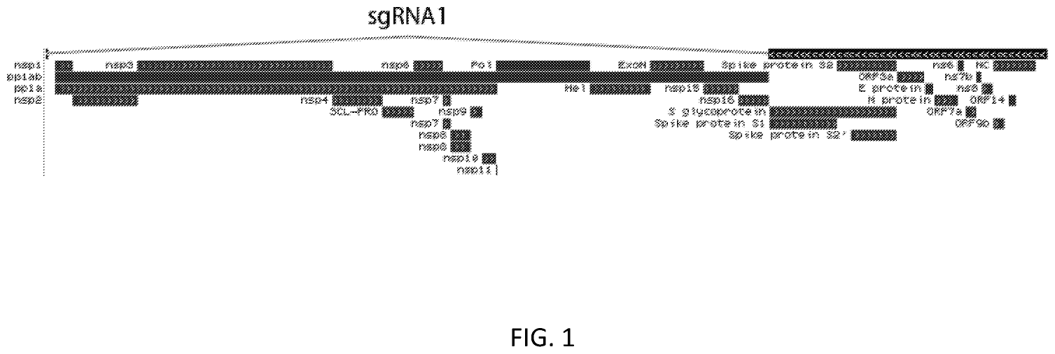

The genome-wide scan for finding efficient RNAi triggers against SAR-COV2 commenced with an initial step of designing shRNAs for the SARS-CoV-2 genome. Since the virus' genome is a positive strand RNA and the structural genes must go through a negative strand intermediary to be translated, the negative strand could be serving as an additional target. Accordingly, the entire 30.3 Kb of the SARS-CoV-2 genome and the additional 8.3 Kb of the negative strand (sgRNA1) that encompass all of the structural genes with the leader sequence were considered potential targeted sequences (see FIG. 1).

The SARS-CoV-2 genome and the negative strand (sgRNA1) were then divided into a series of potential shRNA targets. This process was conducted by parsing the genome into overlapping windows, each having a length of 50 nt with one nucleotide shift. The targeted sequence for the shRNA is a stretch of 22 nt in the middle of the 50 nt window and the rest of the sequence provide the local context.

Multiple filters were then applied on the collections of potential shRNA targets so as to remove regions that cannot be synthesized well, are not even minimally conserved between the viral strains, have sequence attributes that are typically associated with poor shRNA response, and whose guide shRNA seed region can potentially match a human transcript, as outlined in Table 1 below.

TABLE 1
Selection of potential siRNAs

| | # of siRNA | # of siRNA |
|---|---|---|
| | targets on the | targets on the |
| | SARS-CoV-2 | negative strand |
| | plus strand | sgRNA1 of |
| Condition | genome | SARS-Cov-2 |
| Initial number of targets | 29,881 | 8,384 |
| . . . and no restriction sites for EcoRI, MluI, XhoI, | 28,403 | 7,735 |
| and MfeI1⁽¹⁾ | | |
| . . . and no sites with homopolymers of >4nt | 23,162 | 5,979 |
| across the oligo⁽²⁾ | | |
| . . . and between nine to eighteen A/U | 22,870 | 5,850 |
| nucleotides in guide strands⁽³⁾ | | |
| . . . and first guide position is A or U⁽³⁾ | 14,024 | 3,493 |
| . . . and no matches to the human | 13,193 | 3,278 |
| transcriptome⁽⁴⁾ (match = up to 1nt difference in the | | |
| seed region or up to 2nt difference in the entire | | |

| guide to a human transcript) | |
|---|---|
| Total passed | 16,471 |

As seen from Table 1, in total 16,471 potential shRNAs against the SARS-CoV-2 genome and sgRNA1 negative strand were found.

In addition, 1118 control shRNAs were added to the pool of siRNAs. The control siRNAs included siRNAs previously identified as being hyper-potent or poorly potent shRNAs thus enabling tuning the system when evaluating each condition.

In total, a library of 17,589 shRNAs was created.

The 17,589 shRNAs and their corresponding 50 nt target regions were synthesised using a DNA oligo pool (SensAI construct).

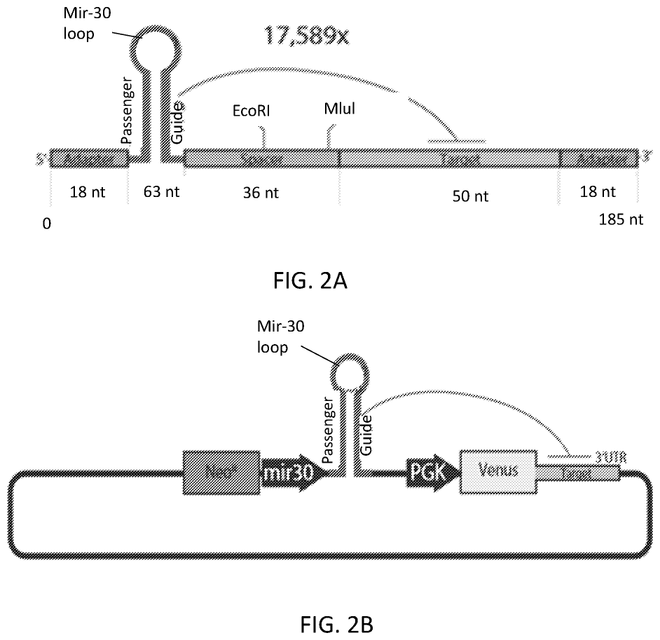

Each of the oligos (outlined in FIG. 2A) had a length of 185 nt and included:
Two PCR annealing sites,
a mir-30 based shRNA with a guide strand (including the potential shRNA identified) and passenger strand,
a spacer with cloning sites (may be preplaced with a reporter), and
a 50 bp region that recapitulates the target site with the genomic context (the target sequence).

A series of cloning steps were then used to introduce a reporter gene (here Venus) into the spacer region, such that the 3′UTR of Venus included the 50 bp target region.

The constructs for each of the shRNAs were then cloned into lentiviral vectors, to form a lentiviral SensAI vector library (FIG. 2B).

Optionally, a 6 bp random sequence was added before the 50 bp target region so that each viral integration obtained its unique barcode.

The lentiviral vector library was used to infect 293 FT cells. Infected cells were selected using a negative selection marker present in the vector. The target multiplicity of infection was 12% to reduce the chance of more than one lentiviral integration per cell, and in total 1.2 million founder cells were infected.

### Example 2—Screening for Hyper-potent shRNA Against SARS-CoV-2

The readout of the screen is Venus expression.

Ideally, a potent shRNA shall cut the target site (the targeted sequence) in the 3′UTR of the Venus mRNA, which will result in a low Venus expression and a dim Venus fluorescent signal. Poorly potent shRNA, on the other hand, will not be able to target the sequence and the cells will exhibit a bright Venus fluorescent signal.

However, one complication is position effect variegation, in which constructs with poorly potent shRNAs integrate into a region with diminished expression. The result of such integration is low Venus expression irrespective of shRNA efficiency, consequently leading to false positives.

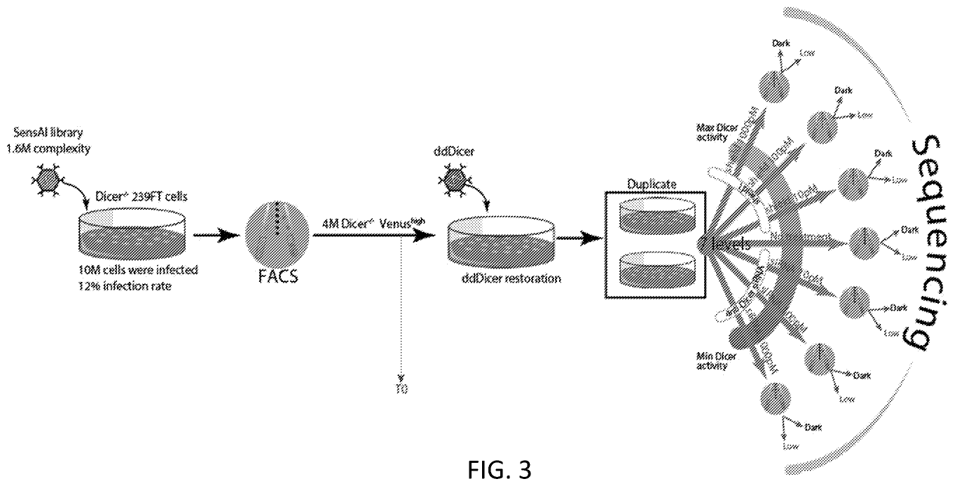

In view of the above deficiency, a multi-step screening procedure to reduce the expression variability was established (outlined in FIG. 3).

Initially the lentiviral vector library was inserted into a 293FT cell line engineered to be Dicer^{null/null} (using CAS9/CRISPR knockout). The absence of Dicer prevents the maturation of the shRNA, meaning that Venus expression initially is independent of the potency of the shRNA.

Three days post infection, 4 million Venus^{high} Dicer^{null/null} 293FT cells out of 50 million cells were collected, using FACS, thereby obtaining cells having the SensAI construct integrated into genomic loci that permit Venus expression.

Subsequently, the expression of Dicer was restored by introducing an exogenous Dicer, thereby recoupling Venus expression to the potency of the shRNA.

The synthetic Dicer was a fusion between human Dicer and a destabilizing domain (ddDicer) that was based on a mutant human FKBP12 protein, enabling tittering the downregulation mediated by the shRNA. The destabilizing domain targets the fused protein to the proteasome, but it is possible to rescue the fused protein using Shield-1, a highly permeable non-toxic small molecule that binds to FKBP12 and stabilizes it (as essentially described in Banaszynski, LA et al. Cell, 2006, 126(5): 995-1004). Using this strategy, the expression of Dicer can be controlled by introducing higher levels of Shield-1.

As an alternative strategy, an siRNA against Dicer was used to dial-down expression.

The main idea behind these various conditions was to find a regime for the RNAi machinery in which hyper-potent shRNAs will still be able to inhibit their targets, while less potent shRNA will cause little if any inhibition.

The lentiviral vector library was subsequently screened in 7 different conditions of ddDicer expression all normalized to cells in which no Dicer was expressed (reflecting the initial relative abundance of the various shRNAs T₀).

The other 7 conditions were:
(i) post ddDicer introduction, treatment with either 10 pM, 100 pM, or 1000 pM of Shield-1
(ii) post ddDicer introduction, followed by treatment with either 10 pM, 100 pM, or 1000 pM of the anti-Dicer siRNA, and
(iii) post ddDicer introduction, without any other treatment.

Each of these seven conditions were conducted with two biological replicates.

Following treatment, the cells in each replicate were sorted into 3 cell populations, based on their Venus expression, namely high, low, and dark.

The cells exhibiting low to dark Venus expression were sequenced. Overall, (2_{[replicates]}×2_{[sorting bins]}×7_{[Dicer Expression levels]}+1_{[T0]}=) 29 sequencing libraries were obtained with Illumina MiSeq with 150 bp paired-end reads, each of which obtained 36 million reads on average. The 50 bp region that corresponds to the target from these libraries were then parsed, annotated back to their shRNA, and the number of unique appearances of each shRNA in each condition counted.

Next, the internal controls were utilized to find the optimal parameters that separates hyper-potent shRNA molecules from the rest of the library. Initially, the relative expression level of each shRNA molecule in one of the post-Dicer screening conditions was compared to its baseline in T₀. To this end, DESeq2 was used while taking into account the two biological replicates to remove some of the noise. For each of the conditions, the area under the curve (AUC) separating the poorly potent from the hyper-potent controls using the DESeq2 enrichment statistic was calculated.

Cells with low Venus expression substantially outperformed the dark cells in terms of separating hyper-potent cells from poorly potent shRNA. In addition, the Shield conditions performed better than no Shield conditions, with 10 pM and 100 pM as the best ones. After further optimization of the sequencing coverage, these two conditions had an AUC of close to 80% for the internal controls. More importantly, these two conditions displayed perfect positive predictive value for the internal controls when restricting the recall to the top 5% of the list. This is especially important since the goal typically is not to find all hyper-potent siRNAs, but to find a handful of potent ones, that can serve as a basis for potential therapeutics.

After finding the optimal parameters, the SARS-CoV-2 shRNAs were ranked in a similar process as that employed for the internal controls, resulting in a list of shRNAs ranking the most likely potent shRNA to the least likely.

### Example 3—Validating the Screen Results

In order to validate the ranking, the consistency of the SARS-CoV-2 shRNA test statistic between the two top conditions was evaluated. This analysis found a Pearson correlation of 72.2% (p<10⁻⁹) between reported statistics of the two conditions, showing that the screen was of significant internal consistency.

Interestingly, in line with previous studies which have reported that highly potent shRNA are typically associated with the absence of adenine in the 20th position of the guide strand, the top shRNAs recovered by the screen were indeed virtually all devoid of Adenine at their 20th position.

10 candidates were chosen for further experimentation (Table 2).

| | | | | | SARS | Strains with |
|---|---|---|---|---|---|---|
| | | | | | COV | a perfect |
| | | | | | strain | match to |
| | Strand | | | Average | conserva- | SARS- |
| Label | +/− | Position | Gene | score | tion | COV2 |
| S1 | + | 6989 | nsp3 | 3.18 | No | 99.9% |
| S2 | + | 9067 | nsp4 | 2.97 | No | 99.9% |
| S3 | + | 23646 | Spike | 2.87 | No | 99.8% |
| S4 | + | 24610 | Spike | 2.24 | Yes | 99.9% |
| S5 | + | 27611 | ORF7A | 2.77 | No | 99.3% |
| S6 | + | 15528 | RDRP | 2.58 | No | 99.9% |
| S7 | + | 15819 | RDRP | 1.82 | Yes | 99.9% |
| S8 | − | 25371 | Spike | 1.79 | Yes | 99.9% |
| S9 | + | 28969 | N | 2.23 | Yes | 99.8% |
| S10 | − | 27511 | ORF7A | 1.87 | Yes | |

Five candidates, namely S1-S4 and S6, were picked mainly based on their high average screen score. The other five candidates (S5 and S7-S10) were additionally characterized by having a 22mer region fully conserved between SARS and SARS-CoV-2, and thus potentially applicable for other beta-coronaviruses as well.

Overall, all ten candidates had perfect matches (>99%) to the SARS-CoV-2 viral strains published in the NCBI database (txid: 2697049) as of October 2020.

Next, the 10 candidates were evaluated using a reporter assay. A 150 nt region around the target site (i.e. around the targeted sequence) of each candidate siRNAs was cloned into the 3′UTR of mCherry.

293 FT cells were then transfected, using lypofectamin 3000 with a combination of the mCherry vector (with the target region) and with a relevant of the 10 candidate siRNA molecules. The following concentrations of siRNA were used: 0 pM (no siRNA), 10 pM, 100 pM, 1 nM, and 10 nM. An eGFP expression vector was added to the transfection mix in order to enable transfection efficiency evaluation. Each experiment was repeated twice. Six control experiments with no eGFP and mCherry vectors were utilized as controls to assess experimental noise.

Flow cytometry was then utilized to measure mCherry and eGFP expression at a single cell resolution, wherein GFP positive cells are indicative of transfected cells (first gating) and mCherry expression indicative of siRNA potency (low to no mCherry expression indicative of a potent siRNA—second gating). The Cherry signal was normalized according to eGFP expression to obtain a median normalized mCherry expression for each condition.

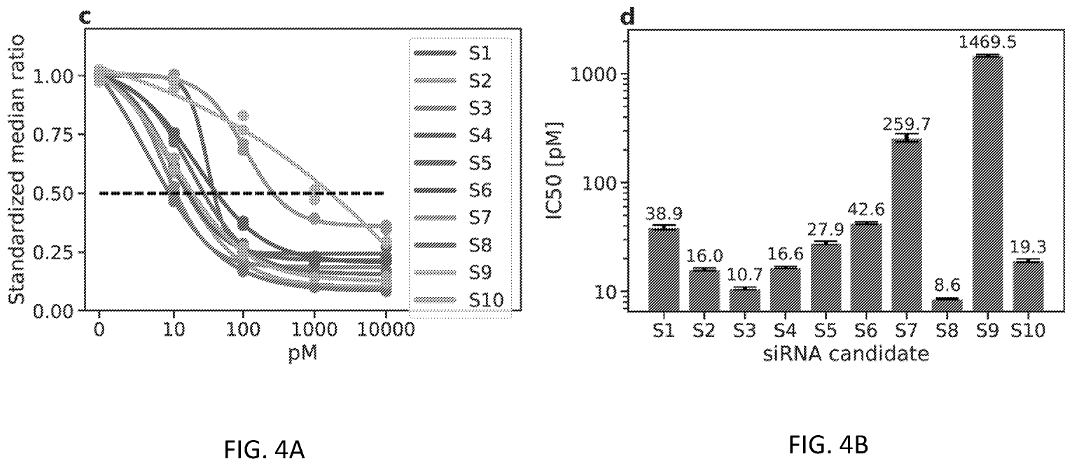

Out of the 10 siRNAs, 8 siRNAs with an IC50 at a concentration of below 50 pM were identified FIG. 4A. In fact, S2, S3, S5, S8, and S10 showed an IC50 of less than 30 pM FIG. 4B, suggesting them being highly potent siRNAs. Only one candidate, S9, showed a relatively poor IC50 in the reporter assay experiment with an IC50 of>1400 pM.

These results clearly demonstrate the ability of the hereindisclosed SensAI assay to identify highly potent siRNAs.

### Example 4—Using Live Virus to Test siRNA Candidates

Next, nine out of the ten candidates were evaluated for their ability to attenuate live SARS-CoV-2.

Initially, Vero E6 cells were transfected with 100 nM of each candidate siRNA in triplicates or with a mock siRNA against eGFP as a negative control. Controls of either no siRNA or eGFP siRNA were used to measure viral load at inoculation.

In order to test the prophylactic capacity of the siRNAs, the cells were transfected 24 hours prior to inoculation with 600 or 6000 TCID50 of live siRNA-CoV-2 for 48 hours.

After the 48 hours, viral load was evaluated by aliquoting 20 ul of the media and quantifying viral RNA using qPCR. To this end, the commercial kit of Seegene Allpex 2019-nCov that measures the RDRP, E, and the N gene simultaneously were utilized.

Advantageously, most of the siRNAs of the hereindisclosed SensAI screen were able to dramatically lower the amount of viral RNA. This as compared to cells without any siRNA treatment, in which the level of SARS-CoV-2 RNA increased by over 100-fold after 48 hours post infection.

Specifically, the siRNA of 5 siRNA, namely S2, S3, S5, S6, and S9 were particularly effective in that SARS-CoV-2 RNA levels were less than 5% than that of untreated cells.

Interestingly, both S8 and S10 showed weak responses (SARS-CoV-2 RNA levels of ˜90% compared to the no siRNA level) despite the high potency in the reporter assay. Noteworthy, these two candidates target the negative stand of SARS-COV2, which is an intermediary for replication. indicating that targeting this intermediary RNA molecule may not sufficiently interfere with viral replication.

Moreover, S9 showed a strong viral inhibition despite its relatively weak result in the reporter assay, suggesting that targeting the N gene may induce a strong response.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1-33. (canceled)

34. A method for retrieving potent RNAi triggers sequences, the method comprising:
a. providing a cell population comprising at least one genetic alteration in the RNAi biogenesis machinery that substantially decreases the maturation of RNAi triggers expressed from the cell's genome;
b. expressing in the cell population a plurality of nucleic acid constructs, each construct comprising: a nucleic acid sequence encoding a candidate RNAi trigger targeting a target gene, the down-regulation of which is desired, wherein the plurality of constructs differ in the sequence of the RNAi trigger;
c. selecting cells expressing the target gene;
d. rescuing the genetic alteration in the RNAi machinery; and
e. identifying cells expressing a potent RNAi trigger by identifying cells which are no longer expressing the target gene or which express only low levels of the target gene, as compared to the expression level of the target gene prior to rescuing procedure.

35. The method of claim 34, wherein the nucleic acid construct further comprises a nucleic acid encoding the target sequence, wherein the target sequence is transcriptionally fused to a nucleotide sequence encoding a reporter gene.

36. The method of claim 35, wherein the selecting of cells expressing the construct comprises selecting cells expressing the reporter gene; and wherein the identifying of cells which are no longer expressing the target gene comprises identifying cells no longer expressing the reporter gene, or which express only low levels of the reporter gene, as compared to the expression level of the reporter gene prior to the rescuing procedure.

37. The method of claim 34, further comprising a step of identifying the sequences of the RNAi triggers expressed by the identified cells.

38. The method of claim 34, wherein the RNAi trigger is an shRNA comprising a guide strand potentially targeting the target gene.

39. The method of claim 38, wherein the guide strands of the plurality of shRNAs overlappingly cover an mRNA or a UTR of a gene of interest.

40. The method of claim 38, further comprising a step of selecting suitable guide strands by discarding sequences that cannot be synthesized well, sequences that have sequence attributes that are typically associated with poor shRNA response, sequences whose guide shRNA seed region can potentially match a human transcript or any combination thereof.

41. The method of claim 38, wherein the target exhibits partial complementarity to the guide sequence.

42. The method of claim 34, wherein the construct further comprises a unique barcode sequence.

43. The method of claim 34, wherein the genetic alteration in the RNAi biogenesis is a Dicer deletion and wherein rescuing the genetic alteration in the RNAi machinery comprises expressing a Dicer gene in the cell.

44. The method of claim 43, wherein the exogenous Dicer is a modulated Dicer and wherein the modulated Dicer comprises a destabilizing domain (ddDicer).

45. The method of claim 44, further comprising a step of treating the cells expressing the ddDicer with a predetermined concentration of Shield-1 molecules, thereby at least partially stabilizing the ddDicer.

46. The method of claim 34, further comprising treating the cells with a predetermined concentration of an shRNA targeting the exogenous Dicer, thereby controllably reducing Dicer activity in the cells.

47. The method of claim 34, further comprising:
introducing a same or a different genetic alteration into the identified cells to thereby substantially decrease the expression of the RNAi trigger;
selecting cells that express the target gene;
rescuing the same or the different genetic alteration; and
identifying cells expressing a potent RNAi trigger by identifying cells which no longer express the target gene or which express only low levels of the target gene, as compared to the expression level of the target gene prior to the rescuing procedure.

48. A kit comprising:
a. a first vector comprising
i. a nucleic acid sequence encoding a candidate RNAi trigger comprising a guide strand potentially targeting a target sequence, the down-regulation of which is desired, wherein the target sequence comprises at least part of an mRNA or a 3′UTR of a target gene; and
ii. a nucleic acid sequence encoding the target sequence, wherein the target sequence is transcriptionally fused to a nucleotide sequence encoding a reporter gene; and
b. a second vector comprising:
i. a nucleic acid sequence encoding an exogenous Dicer, wherein the exogenous Dicer is a modulated Dicer having impaired functionality.

49. The kit of claim 48, wherein the kit further comprises a molecule configured to stabilize the modulated Dicer, thereby restoring Dicer functionality, wherein the molecule is Shield-1.

50. The kit of claim 48, wherein the target sequence targets a SARS-COV2 transcript.

51. The kit of claim 48, wherein the guide strand is devoid of Adenine at position 20.

52. A cell line expressing:
a. a nucleic acid sequence encoding a candidate RNAi trigger comprising a guide strand potentially targeting a target sequence, the down-regulation of which is desired; and
b. a nucleic acid sequence encoding the target sequence, wherein the target sequence is transcriptionally fused to a nucleotide sequence encoding a reporter gene,
wherein the cell line comprises a genetic alteration in the RNAi biogenesis machinery.

53. The cell line of claim 52, wherein the genetic alteration in the RNAi biogenesis is a Dicer deletion.
